SEQUENCE LISTING (6 sequences)

SEQ ID NO: 1 - protein, 9 aa, Artificial Sequence
  Synthetic
GWGGRGDSP

SEQ ID NO: 2 - protein, 9 aa, Artificial Sequence
  Synthetic
GWGGRGESP

SEQ ID NO: 3 - protein, 8 aa, Artificial Sequence
  Synthetic
GGRGDSPK

SEQ ID NO: 4 - protein, 5 aa, Artificial Sequence
  Synthetic
RGDFC

SEQ ID NO: 5 - protein, 14 aa, Artificial Sequence
  Synthetic
CGKKQRFRHRNRKG

SEQ ID NO: 6 - protein, 8 aa, Artificial Sequence
  Synthetic
KRTGQYKL

(19) United States
(12) Patent Application Publication
(10) Pub. No.: US 2014/0273221 A1
(43) Pub. Date: Sep. 18, 2014

(54) SUBSTRATES FOR GENERATION OF CONTROLLED HUMAN PLURIPOTENT STEM CELL COLONY SIZE AND SHAPE

(71) Applicant: WISCONSIN ALUMNI RESEARCH FOUNDATION, Madison, WI (US)

(72) Inventors: Justin T. Koepsel, Madison, WI (US); William L. Murphy, Waunakee, WI (US); Sheeny K.L. Levengood, Bothell, WA (US); Stefan Zorn, Madison, WI (US)

(73) Assignee: WISCONSIN ALUMNI RESEARCH FOUNDATION, Madison, WI (US)

(21) Appl. No.: 13/835,102

(22) Filed: Mar. 15, 2013

(51) Int. Cl. C12N 5/0735

(52) U.S. Cl. CPC C12N 5/0606
USPC 435/379; 435/378

(57) ABSTRACT

Methods of cell culture using patterned SAM arrays are disclosed. Advantageously, the disclosed methods use SAM arrays presenting adhesion peptides to grow confluent monolayers that can invaginate to form an embryoid body.

### STATEMENT OF GOVERNMENT SUPPORT

This invention was made with government support under EB005374 awarded by the National Institutes of Health and NIGMS 5 T32 GM08349 awarded by the National Institutes of Health Training Program. The government has certain rights in the invention.

### INCORPATION OF SEQUENCE LISTING

A paper copy of the Sequence Listing and a computer readable form of the Sequence Listing containing the file named “28243-170 (P120127US01)_ST25.txt”, which is 1,511 bytes in size (as measured in MS-DOS), are provided herein and are herein incorporated by reference. This Sequence Listing consists of SEQ ID NOs: 1-6.

### BACKGROUND OF THE DISCLOSURE

The present disclosure relates generally to the culture of stem cells. More particularly, the present disclosure relates to cell culture methods for generating colonies of stem cells having controlled size.

The substrate on which cells are cultured is important for successful cellular growth and tissue generation. For example, it has been demonstrated that attachment to the substrate by human embryonic stem cells may contribute to the variability in whether the cells remain undifferentiated or undergo differentiation.

Many protocols for differentiation of pluripotent stem cells begin with the formation of 3-dimensional aggregates of cells called embryoid bodies (EBs).

Methods for forming embryoid bodies involve techniques such as scraping adherent ES cell and induced pluripotent stem cell cultures and mild treatment with proteases such as trypsin and/or dispase to release large clumps of cells, followed by placing the resulting aggregates in non-adherent suspension culture. The aggregates formed using these methods are heterogeneous in size and shape, which can lead to inefficient and uncontrolled differentiation. Aggregate size can also directly affect subsequent differentiation pathways. To address these issues, cell culture substrates such as multi-well plates with wells having defined widths have been developed. Another method creates dots of a substrate material such as Matrigel® onto the surface of a plate.

Self-assembled monolayers (“SAMs”) in array formats (i.e., SAM arrays) have been constructed that present ligands to cells plated onto the array. A SAM array is an organized layer of amphiphilic molecules in which one end of the molecule exhibits a specific, reversible affinity for a substrate and the other end of the molecule has a functional group. Because the molecule used to form the SAM array is polarized, the hydrophilic “head groups” assemble together on the substrate, while the hydrophobic tail groups assemble far from the substrate. Areas of close-packed molecules nucleate and grow until the surface of the substrate is covered in a single monolayer. The use of alkanethiols to construct SAM arrays allow for the formation of reproducible SAM arrays and surfaces. SAM arrays may be used to identify specific ligands or epitopes that promote cellular attachment, spreading, proliferation, migration and differentiation, as well as for modulating these cellular activities differentially on each spot on the same SAM array.

Aggregate size and shape can also directly affect subsequent differentiation pathways and lead to inefficient and uncontrolled differentiation. Accordingly, there exists a need for alternative substrates and methods to control the size and/or shape of colonies as well as avoid treatments such as scraping and enzymes used to harvest the cell aggregates.

### SUMMARY OF THE DISCLOSURE

The present disclosure relates generally to the culture of cells. More particularly, the present disclosure relates to cell culture methods for generating colonies of cells having controlled size. It has been found that cell colony size may be controlled in cell culture via SAM arrays with controlled spot size.

In one aspect, the present disclosure is directed to a method of controlling the formation of a cell culture aggregate. The method comprises: culturing a cell on a self-assembled monolayer (“SAM”) array spot for a sufficient time to form a confluent monolayer of cells; and detaching the confluent monolayer of cells. The method can further comprise culturing the confluent monolayer for a sufficient time to allow the monolayer to invaginate.

In another aspect, the present disclosure is directed to a method of preparing a cell aggregate of a uniform size. The method comprises: culturing a cell on a self-assembled monolayer (“SAM”) array spot of a specified diameter for a sufficient time to form a confluent monolayer of cells; detaching the confluent monolayer of cells; collecting the confluent monolayer of cells; and placing the resulting confluent monolayer of cells in non-adherent suspension culture.

In another aspect, the present disclosure is directed to a method of preparing a cell aggregate of a specified shape. The method comprises: culturing a cell on a self-assembled monolayer (“SAM”) array spot of a specified shape for a sufficient time to form a confluent monolayer of cells; detaching the confluent monolayer of cells; and collecting the confluent monolayer of cells.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The disclosure will be better understood, and features, aspects and advantages other than those set forth above will become apparent when consideration is given to the following detailed description thereof. Such detailed description makes reference to the following drawings, wherein:

FIG. 1 is a schematic illustrating the steps for preparing a self-assembled monolayer array used in the methods of the present disclosure.

FIG. 2A depicts hESC (H1) monolayer formation as a function of density of adhesion ligands (cyclic RGD) as described in Example 1.

FIG. 2B depicts hESC (H1) monolayer formation as a function of adhesion ligands as described in Example 1.

FIG. 2C depicts hESC (H1) monolayer formation as a function of adhesion ligands as described in Example 1.

FIG. 3A depicts hESC (H1) monolayer formation as a function of spot size as described in Example 1.

FIG. 3B depicts hESC (H1) monolayer formation as a function of spot size as described in Example 1. More particularly, FIG. 3B depicts invagination of circle-shaped hESC monolayers.

FIG. 3C depicts hESC (H1) monolayer formation as a function of spot shape as described in Example 1.

FIG. 3D depicts hESC (H1) embryoid body formation as a function of spot shape as described in Example 1. More particularly, FIG. 3D depicts invagination of oval and cross-shaped hESC monolayers.

FIG. 3E depicts rapid embryoid body formation from 4-14 hours after hESC (H1) seeding on a spot with 5% ligand density, functionalized with a 1:1 mixed layer of cyclic RGD and CGKKQRFRHRNRKG as described in Example 1.

FIG. 4 depicts hESC (H1) monolayer formation as a function of cell lineages as described in Example 1.

FIGS. 5A-D depict pluripotency staining of hESC (H1) grown on SAM array for Oct3/4 and Nanog as described in Example 1.

FIGS. 6A-D depict staining of hESC (H1) grown on SAM array for Oct3/4 and Nanog at Day 0 as described in Example 1.

FIGS. 7A-D depict staining of hESC (H1) grown on SAM array for Oct3/4 and Nanog at Day 1 as described in Example 1.

FIGS. 8A-D depict staining of hESC (H1) grown on SAM array for Oct3/4 and Nanog at Day 2 as described in Example 1.

FIGS. 9A-D depict staining of hESC (H1) grown on SAM array for Oct3/4 and Nanog at Day 3 as described in Example 1.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein may be used in the practice or testing of the present disclosure, the preferred materials and methods are described below.

In accordance with the present disclosure, methods for preparing colonies of stem cells with controlled size and/or shape have been discovered. More particularly, the present disclosure relates to methods for preparing stem cell colonies with controlled size and/or shape using SAM arrays. It has been found that stem cell colony size and/or shape may be controlled in cell culture via SAM arrays with controlled spot size and/or shape.

In one aspect, the present disclosure is directed to a method of controlling the formation of a cell culture aggregate. The method comprises culturing a cell on a spot (also referred to herein as “an array spot”) of a self-assembled monolayer (“SAM”) array for a sufficient time to form a confluent monolayer of cells and detaching the confluent monolayer of cells. As known by those skilled in the art, the initial density of the cells can influence the time to confluence. A particularly suitable seeding density can be, for example, 10⁵ cells/cm², in which cells can reach confluence in a range of between about 12 hours to about 36 hours. A particularly suitable time period after which cells can be detached can be, for example, about 36 hours to about 84 hours. More particularly, for 10⁵ cells/cm², cells can be detached at a time period of from about 36 hours to about 60 hours after initial seeding. For larger colonies such as, for example, an area greater than about 7 mm², detachment can require up to about 84 hours. The method may further comprise culturing the confluent monolayer for a sufficient time to allow the monolayer to invaginate. As used herein, “invaginate” or “invagination” or “invaginating” refer to the monolayer lifting from the surface of the SAM array and folding into a cell aggregate. In one embodiment, invagination of the monolayer can occur at a time of from about 48 hours to about 72 hours when the density of seeded cells is 10⁵ cells/cm². In another embodiment, invagination of the monolayer can occur at a time of from about 6 hours to about 72 hours by varying the ligand density from about 2% to about 10%. In another embodiment, invagination of the monolayer can occur at a time of from about 24 hours to about 72 hours by varying the diameter of the array spot size. Suitable array spot diameter size can be from about 600 μm to about 6 mm. A particularly suitable array spot diameter size can be from about 1.2 mm to about 2.4 mm. The method may further comprise collecting the cells after the cells are detached from the SAM array and/or an array spot.

Self-assembled monolayer (SAM) arrays are known in the art. Suitable SAM arrays include patterned SAM arrays. Patterned SAM arrays are those that have been developed to spatially localize ligands to create spatially and chemically-defined spots or islands created to promote cell attachment within the spot. Methods for preparing patterned SAM arrays can be, for example, those prepared by microcontact printing methods, microfluidics approaches, stamping, photochemistry with micro-patterned photomasks, and locally destroying/removing regions of a fully formed SAM and reforming new SAMs in the destroyed regions. Particularly suitable self-assembled monolayer arrays useful for the methods of the present disclosure are those described in U.S. patent application Ser. No. 13/465,120, and incorporated by reference herein in its entirety. Briefly, SAM arrays are prepared by adhering a polymer stencil to a metal-coated substrate. The polymer stencil includes at least one well. A solution of alkanethiolates bearing oligo (ethylene glycol) groups is added to each well of the stencil. Carbodiimide chemistry is used to covalently immobilize at least one cell adhesion peptide to the oligo (ethylene glycol) bearing alkanethiolates. An alkanethiolate self-assembled monolayer spot that presents a cell adhesion peptide is formed on the substrate in each well of the polymer stencil. The polymer stencil is then removed from the substrate to reveal a self-assembled monolayer spot on the substrate. The substrate is then backfilled with hydroxyl terminates alkanethiolates to form a second self-assembled monolayer that surrounds each alkanethiolate self-assembled monolayer spot. Use of alkanethiolate-bearing oligo (ethylene glycol) groups promotes specific protein-surface interactions, while backfilled regions with hydroxyl terminates surrounding each array spot generates an inert surface that prevents and/or hampers protein-surface and cell-surface interactions within the backfilled region.

Once a self-assembled monolayer array is prepared, the method includes contacting (“seeding”) a cell with the self-assembled monolayer array. Single cell suspensions can be directly contacted with an array spot. Because of the array features described herein, a single cell suspension solution can also be applied to an entire SAM array. Cells that come in contact with an array spot that presents a surface that promotes cell adhesion and growth will adhere to the array spots, whereas cells that come in contact with the backfilled region will not adhere. After a time sufficient to allow cells to adhere to the array spots (e.g., about 12 hours to 36 hours for 10⁵ cells/cm²), the SAM array can be washed with fresh culture medium (or another buffer) to remove unattached cells.

The cells are cultured on the arrays to form a confluent monolayer for a time that is sufficient for the cells to fill the area defined by the array spot. One skilled in the art can monitor whether cells fill the area using microscopy to directly observe cells on the arrays. A sufficient amount of time can be, for example, from about 12 hours to about 36 hours. The density of cells in the cell suspension that is used to seed the SAM array can increase or decrease the time that is sufficient for the cells to fill the area (i.e., form a confluent monolayer) defined by the array spot. If a low density of cells is used to seed the entire SAM array, for example, it can take the cells a longer length of time to proliferate to a colony size that fills the area. In contrast, if a high density of cells is used to seed the entire SAM array, for example, it can take the cells a shorter length of time to proliferate to a colony size that fills the area. Additionally, the type of cell that is used to seed the array or the array spot can determine the time needed to fill the area defined by the array spot. If the cell type that is used has a fast proliferation rate, for example, it can take the cells a shorter length of time to proliferate to a colony size that fills the area. In contrast, if the cell type that is used has a slow proliferation rate, for example, it can take the cells a longer length of time to proliferate to a colony size that fills the area. One skilled in the art can, without undue experimentation, determine the time that is sufficient for a specific cell type to form a confluent monolayer that fills the area defined by the array spot by seeding arrays or array spots and monitoring cell growth by microscopy, for example. One skilled in the art can, without undue experimentation, determine the time that is sufficient for a specific density of cells to be seeded to an array or array spot to form a confluent monolayer of cells that fills the area defined by the array spot by seeding arrays or array spots with different solutions containing different densities of cells and monitoring cell growth by microscopy, for example.

The method further includes detaching the confluent monolayer. The confluent monolayer can be detached from the SAM by mechanical perturbations. Suitable mechanical perturbations may be by gentle fluid shearing by pipetting culture medium over the colonies to dislodge the colonies. Another suitable method for detaching the confluent monolayer can be, for example, by gently tapping or bumping the substrate. Additionally, the confluent monolayer may be detached by monitoring the confluent monolayer for a sufficient time and collecting colonies that spontaneously detach from the substrate.

Upon detachment, colonies may further be collected. Colonies may be collected by aspirating the colonies from the medium. Additionally or alternatively, the media may be obtained and colonies collected by allowing colonies to settle by gravity or be collected by centrifugation.

In another aspect, the present disclosure is directed to a method of preparing a cell aggregate of a uniform size. The method comprises culturing a cell on a self-assembled monolayer (“SAM”) array spot of a specific diameter for a sufficient time to form a confluent monolayer of cells; detaching the confluent monolayer of cells; and collecting the confluent monolayer of cells. The method can further comprise placing the collected confluent monolayer of cells in non-adherent suspension culture.

The SAM array may be prepared as described herein or using other methods known by those skilled in the art to prepare a SAM array having array spots in which the method allows for controlling array spot size. Array spot size can be any desired size. Particularly suitable array spot size can be, for example, at least 400 μm, including from about 600 μm to about 6 mm.

In another aspect, the present disclosure is directed to a method of preparing a cell aggregate of a specified shape. The method comprises culturing a cell on a self-assembled monolayer (“SAM”) array spot of a specified shape for a sufficient time to form a confluent monolayer of cells; detaching the confluent monolayer of cells; and collecting the confluent monolayer of cells. The method can further comprise placing the collected confluent monolayer of cells in non-adherent suspension culture. The method can further comprise analyzing the confluent monolayer of cells.

The SAM array may be prepared as described herein or using other methods known by those skilled in the art to prepare a SAM array having array spots in which the method allows for controlling array spot shape. Array spot shape can be any desired shape as known in the art. Particularly suitable array spot shapes can be, for example, circular, oval, oval cross, star, and hand shaped spots. Spot shape can be used to control time to invagination. For example, a circular spot shape can increase the time it takes for cell monolayers to begin invaginating. Spots in the shape of oval or oval cross-shape, for example, can decrease the time it takes for cell monolayers to begin invaginating.

Cells can be seeded on SAM arrays or array spots as described herein. The cells are cultured on the arrays to form a confluent monolayer for a time that is sufficient for the cells to fill the area defined by the array spot. The shape of the confluent monolayer will correspond to the shape of the array spot. Once the confluent monolayer attains a shape defined by the array spot shape, the method further includes detaching the confluent monolayer as described herein. The confluent monolayer can then be collected as described herein. The collected confluent monolayer can then be placed in a non-adherent suspension culture.

Confluent monolayers and/or cells can be further processed by further culturing cells in a non-adherent suspension culture. Cells can also be further be analyzed by microscopy, for gene expression, protein expression, and combinations thereof.

Suitable cells for use in the methods of the present disclosure may be any cell known by those skilled in the art. Particularly suitable cells may be, for example, pluripotent stem cells, mesenchymal stem cells (MSCs), umbilical vein endothelial cells (UVECs), NIH 3T3 fibroblasts, dermal fibroblasts(DFs), fibrosarcoma cells (HT-1080s), and embryonic stem cells (ESCs). Particularly suitable cells may be, for example, human induced pluripotent stem cells, human mesenchymal stem cells (MSCs), human umbilical vein endothelial cells (UVECs), human dermal fibroblasts(DFs), HT-1080s fibrosarcoma cells (HT-1080s), human embryonic stem cells (ESCs) and iPS IMR90-4 cells.

The methods of the present disclosure provide alternative techniques for generating stem cell colonies having controlled size and/or shape. Advantageously, the aggregates formed using these methods are heterogeneous in size and shape, which can lead to more efficient and controlled differentiation of the cells. Because aggregates formed using these methods have a uniform size and shape, better control over which differentiation pathway the cells proceed can also be achieved.

The disclosure will be more fully understood upon consideration of the following non-limiting Example.

### Example

In this Example, a SAM array having an adhesion ligand was prepared.

Carboxylic acid-capped hexa(ethylene glycol) undecanethiole (HS—C₁₁-(O—CH₂—CH₂)₆—O—CH₂—COOH) (referred to herein as “HS—C₁₁-EG₆-COOH”), was purchased from Prochimia (Sopot, Poland). 11-tr(ethylene glycol)-undecane-1-thiol (HS—C₁₁-(O—CH₂—CH₂)₃—OH (referred to herein as “HS—C₁₁-EG₃-OH”) was synthesized as described in (Prime and Whitesides, J. Am. Chem. Soc. 115(23)):10714-10721 (1993)). Fmoc-protected amino acids and Rink amid MBHA peptide synthesis resin were purchased from NovaBiochem (San Diego, Calif.). Hydroxybenzotriazol (HOBt) was purchased from Advanced Chemtech (Louisville, Ky.). Diisopropylcarbodiimide (DIC) was purchased from Anaspec (San Jose, Calif.). N-hydroxysuccinimide (NHS), n-(3-dimethylaminopropyl)-N′-ethylcarbodiimide hydrochloride (EDC), sodium dodecyl sulfate (SDS), trifluoroacetic acid (TFA), diethyl ether, and deionized ultrafiltered water (DIUF H₂O) were purchased from Fisher Scientific (Fairlawn, N.J.). Triisopropylsilane (TIPS), piperidine, dimethylformamide (DMF), acetone, hexanes, and acetonitrile were purchased from Sigma-Aldrich (St. Louis, Mo.). Absolute ethanol (EtOH) was purchased from AAPER Alcohol and Chemical Co. (Shelbyville, Ky.). All purchased items were of analytical grade and used as received. Thin films of 100 Å Au <111>, 20 Å Ti on 1″×3″×0.040″ glass were purchased from Platypus Technologies, LLC (Madison, Wis.).

Standard solid phase Fmoc-peptide synthesis (Fmoc SPPS) was performed to synthesize peptides using a 316c automated peptide synthesizer (C S Bio, Menlo Park, Calif.). Rink amide MBHA resin was used as the solid phase, and HOBt and DIC were used for amino acid activation and coupling. After coupling the final amino acid, a 4-hour incubation in TFA, TIPS, and DIUF (95:2.5:2.5) released the peptide from resin and removed protecting groups. Released peptide was extracted from the TFA/TIPS/DIUF cocktail via precipitation in cold diethyl ether. Lyophilized peptides were analyzed using matrix-assisted laser desorption/ionization-time-of-flight (MALDI-TOF) mass spectrometry with a Bruker Reflex II (Billerica, Mass.). The purity of synthesized peptides was verified to be greater than 90% via HPLC using a C18 analytical column (Shimadzu, Kyoto, Japan) with a gradient of 0-70% H₂O+0.1% TFA/acetonitrile and a flow rate of 0.9 mL/minute. GWGGRGDSP (SEQ ID NO: 1), GWGGRGESP (SEQ ID NO: 2) adhesion and mutant peptides were synthesized with tryptophan-bearing spacers to aid in determination of peptide concentration via UV/Vis. Peptide stocks were prepared at 300 μM in PBS as pH 7.4 as determined by absorbance at 280 nm using extinction coefficients outlined by Gill and von Hippel (Analytical Biochemistry 182(2):319-326 (1989)). Fluorescently-labeled GGRGDSPK (SEQ ID NO: 3) was synthesized as previously described (Koepsel and Murphy, Langmuir 25(21):12825-34 (2009)) and peptide concentration was determined by absorbance of the 5(6)-carboxyfluorescein group at 492 nm using an extinction coefficient of 81,000 cm⁻¹M⁻¹

Polymer stencils containing arrays of wells were created using soft lithography. Master molds containing arrays of 1.2 mm to 2 4 mm diameter posts were fabricated from SU-8 (Microchem, Newton, Mass.) spin-coated silicon wafers using conventional photolithography techniques. Polydimethylsiloxane (PDMS) (Sylgard 184, Dow Corning, Midland, Mich.) was prepared by mixing a 10:1 ratio of base:curing agent (w/w) followed by degassing for ˜30 minutes. The degassed mixture was cast over the mold and cured for 4 hours at 85° C. Following curing, PDMS stencils were removed from molds and cleaned in hexanes using overnight Soxhlet extraction. After cleaning, stencils were placed in vacuo to remove residual solvent from the Soxhlet extraction process.

Gold slides were placed into a 150 mm glass Petri dish, covered with EtOH and sonicated for ˜1 minute using an ultrasonic bath (Bransonic 1510, Branson, Danbury, Conn.). Sonicated gold chips were then rinsed with EtOH and blown dry with N₂. As illustrated in FIG. 1, SAM arrays were fabricated as follows: elastomeric (polymer) stencils containing arrays of 1.2 mm to 2 4 mm diameter holes were placed on a bare gold surface to form an array of wells on the gold substrate. For spot shape, elastomeric stencils with arrays in the shape of circles, ovals, and oval cross were placed on a bare gold surface to form an array of wells having these shapes on the gold substrate. Wells were then filled with 1 mM ethanolic alkanethiolate solution and incubated for 10 minutes in a chamber containing a laboratory wipe soaked in ethanol to prevent evaporation during local SAM formation. Alkanethiolate solutions were then aspirated and wells were rinsed with DIUF H₂O. Carboxylate groups were then converted to active ester groups by adding a solution of 100 mM NHS and 250 mM EDC in DIUF H₂O pH 5.5 to wells and incubated for 10 minutes. After an additional rinse with DIUF H₂O, 300 μM solutions of GWGGRGDSP (SEQ ID NO: 1), GWGGRGESP (SEQ ID NO: 2; glycosaminoglycan peptide), cyclo(RGDF_{D}C) (SEQ ID NO: 4; wherein “F_{D}” denotes D-phenylalanine; commercially available from Peptides International, Louisville, Ky.), CGKKQRFRHRNRKG (SEQ ID NO: 5; commercially available from GenScript, Piscataway, N.J.) or KRTGQYKL (SEQ ID NO: 6; commercially available from GenScript, Piscataway, N.J.) in PBS and pH 7.4 were added to each well and incubated for 1 hour in a humidity controlled chamber to covalently couple peptides to each array spot. After a final rinse in DIUF H₂O, regions surrounding array spots were backfilled with HS—C₁₁-EG₃-OH. This was accomplished by submerging the gold substrate and attached elastomeric stencil in an aqueous 0.1 mM HS—C₁₁-EG₃-OH solution (pH 2.0), removing the stencil, and incubating for 10 minutes. Following backfilling, the array was rinsed with 0.1 wt % SDS in DIUF H₂O, DIUF H₂O, and EtOH and then dried under a stream of N₂. Arrays were stored in sterile DIUF H₂O at 4° C. and used within 24 hours.

Pluripotent stem cells were seeded on arrays at a density of 10⁵ cells/cm². Cells were cultured in E8 medium with ROCK inhibition (using Y-27632) for 12 hours to 36 hours until reaching confluence. Colonies that spontaneously detached from SAM spots were also harvested. Colonies were analyzed for Oct 3/4 and Nanog expression by immunofluorescence using DAPI to stain nuclei.

The concentration of the integrin adhesion peptides GWGGRGDSP (SEQ ID NO: 1) and cyclic RGD (SEQ ID NO: 4) on the array spot was varied between 2% and 10% by the fraction of COOH groups functionalized with peptides present at the spot among background OH functionalities. As shown in FIG. 2A, the density of the adhesion ligand (cyclic RGD; SEQ ID NO: 4) affected hESC monolayer adhesion over a time from 4 hours to 48 hours in culture. At 48 hours in culture, the hESC monolayer formed in the 2% COOH density array spot was loosely associated with the array spot, whereas the hESC monolayers formed in the 5% and 10% density array spots were more strongly adhered to the array spot. These results demonstrated that a COOH fraction of 2% led to a significantly lower cell adhesion as compared to 5% COOH, whereas 10% COOH did not lead to an improved attachment for surfaces functionalized with cyclic RGD. In addition, a lower peptide density leads to an earlier start of the invagination process (see, FIG. 2A, 2% COOH condition).

The particular adhesion ligands used in the array spot also influenced cell monolayer adhesion in the array spot. As shown in FIG. 2B, the best cell adhesion of the hESC monolayer was observed with cyclic RGD (SEQ ID NO: 4) and GWGGRGDSP (SEQ ID NO: 1). No significant differences were observed using the scrambled reference GWGGRGESP (SEQ ID NO: 2). As shown in FIG. 2C, no significant adhesion was observed with the heparin binding peptide KRTGQYKL (SEQ ID NO: 6). After initial attachment of the cells to the glycosaminoglycan binding peptide CGKKQRFRHRNRKG (SEQ ID NO: 5) array spots, the cells detached within 12 h (FIG. 2C). Additionally, significantly less cell attachment was observed on the glycosaminoglycan binding peptide CGKKQRFRHRNRKG (SEQ ID NO: 5) array spots.

The size of the array spots was found to influence monolayer morphology over time. As shown in FIG. 3A, the edges of the hESC monolayers formed on 1.2 mm and 2 4 mm diameter array spots began to fold over after 48 hours in culture. As shown in FIG. 3B, the morphology of hESC monolayers cultured on circle-shaped array spots was followed from a time period of 6 hours to 96 hours. At 72 hours, hESC monolayers cultured on 1.2 mm diameter array spots were in the form of balls similar to embryoid bodies that became tight balls of cells by 96 hours. At 72 hours, the edges of cells from the hESC monolayers cultured on 2 4 mm diameter array spots were still in the process of folding over, but formed tight balls of cells by 96 hours.

As shown in FIG. 3C, the morphology of hESC monolayers cultured on circle-, oval-, and oval cross-shaped array spots was followed from a time period of 4 hours to 48 hours. At 4 hours and 24 hours in culture, the cell monolayers assumed the shape of the array spot. At 48 hours, the edges of the hESC monolayers formed on the circular shaped array spot had just begun to fold over, whereas hESC monolayers formed on the oval-shaped array spot were folded. hESC monolayers formed on oval cross-shaped array spots were formed into a ball-like shape by 48 hours that was reminiscent of an embryoid body. As further shown in FIG. 3D, hESC monolayers formed on the oval-shaped array spot appeared to fold over longitudinally to form an elongated morphology (see 72 hour photomicrograph) before becoming more ball-like at the 96 hour time point. hESC monolayers formed on the oval cross-shaped array spot also appeared to fold along a longitudinal axis at each arm of the cross before becoming ball-shaped at the 96 hour time point.

As shown in FIG. 3E, a mixed layer of the cyclic RGD (SEQ ID NO: 4) and the CGKKQRFRHRNRKG (SEQ ID NO: 5) could be used to influence the time it took for cell monolayers to form the ball-shaped (embryoid body-like) morphology. Specifically, a 1:1 functionalization with cyclic RGD (SEQ ID NO: 4) and CGKKQRFRHRNRKG (SEQ ID NO: 5) at a ligand density of 5% lead to invagination within 16 h after seeding.

To show the universality of the cell culture approach, iPS IMR90-4 cells were grown on array spots. As demonstrated in FIG. 4, iPS IMR90-4 cells also formed monolayers on array spots.

Cells cultured on array spots were stained for pluripotency markers Oct 3/4 and Nanog. Cell nuclei were also stained with DAPI to identify cells. FIGS. 5A-D show overlay images of Oct 3/4, Nanog, and nuclear staining for Days 1-3 to demonstrate pluripotency of the cells at each day. As shown in FIGS. 6A-D, 5 hours after seeding (Day 0), cells stained positive for Oct 3/4 and Nanog. At 24 hours after seeding (Day 1), only cells near the edge of the monolayer stained positive for Oct 3/4 and Nanog (FIGS. 7A-D). At 48 hours after seeding (Day 2), right after the edges of the monolayer began to fold (invaginate), only a part of the cells stained positive for Oct 3/4 and Nanog (FIGS. 8A-D). At 72 hours after seeding (Day 3), no more cells stained positive for Oct 3/4 and Nanog (FIGS. 9A-D). These results demonstrate that as cells develop on the array spot, the morphological changes observed for the cell monolayers correlates with loss of pluripotency markers to form ball-like cells similar to embryoid bodies.

These results demonstrate that the SAM arrays of the present disclosure can be used to culture cells with controlled size and shape. Moreover, the methods of the present disclosure allow for the development of a monolayer of cells that proceeds through morphological stages to develop into 3-dimensional ball-shaped cells similar to embryoid bodies. Further, as the cells develop and go through morphological changes, pluripotency marker staining also indicates that the cells lose their pluripotency during culture.

In view of the above, it will be seen that the several advantages of the disclosure are achieved and other advantageous results attained. As various changes could be made in the above methods without departing from the scope of the disclosure, it is intended that all matter contained in the above description and shown in the accompanying drawings shall be interpreted as illustrative and not in a limiting sense.

When introducing elements of the present disclosure or the various versions, embodiment(s) or aspects thereof, the articles “a”, “an”, “the” and “said” are intended to mean that there are one or more of the elements. The terms “comprising”, “including” and “having” are intended to be inclusive and mean that there may be additional elements other than the listed elements.

What is claimed is:

1. A method of controlling the formation of a cell culture aggregate, the method comprising: culturing a cell on an array spot of a self-assembled monolayer array for a sufficient time to form a confluent monolayer of cells; and detaching the confluent monolayer of cells.

2. The method of claim 1, wherein the confluent monolayer is mechanically detached from the array spot.

3. The method of claim 1, wherein the confluent monolayer is cultured until the confluent monolayer spontaneously detaches from the array spot.

4. The method of claim 3, wherein the confluent monolayer is cultured for a period of from about 36 hours to about 84 hours.

5. The method of claim 1, further comprising culturing the confluent monolayer for a sufficient time to allow the confluent monolayer to invaginate.

6. The method of claim 5, wherein the confluent monolayer is cultured for a period of from about 10 hours to about 72 hours.

7. The method of claim 1, wherein the cell is selected from the group consisting of an induced pluripotent stem cell, a mesenchymal stem cell, an umbilical vein endothelial cell, a dermal fibroblast, a fibrosarcoma cell, an embryonic stem cell, an iPS IMR90-4 cell and combinations thereof.

8. A method of preparing a cell aggregate of a uniform size, the method comprising:
culturing a cell on an array spot of a self-assembled monolayer array spot of a specified diameter for a sufficient time to form a confluent monolayer of cells;
detaching the confluent monolayer of cells; and
collecting the confluent monolayer of cells.

9. The method of claim 8, wherein the specified diameter of the array spot is from about 600 μm to about 6 mm.

10. The method of claim 8, further comprising culturing the confluent monolayer for a sufficient time to allow the confluent monolayer to invaginate.

11. The method of claim 8, wherein the confluent monolayer is mechanically detached from the array spot.

12. The method of claim 8, wherein the confluent monolayer is cultured until the confluent monolayer spontaneously detaches from the array spot.

13. The method of claim 8, wherein the cell is selected from the group consisting of an induced pluripotent stem cell, a mesenchymal stem cell, an umbilical vein endothelial cell, a dermal fibroblast, a fibrosarcoma cell, an embryonic stem cell, an iPS IMR90-4 cell and combinations thereof.

14. A method of preparing a cell aggregate of a specified shape, the method comprising:
culturing a cell on a self-assembled monolayer array spot of a specified shape for a sufficient time to form a confluent monolayer of cells;
detaching the confluent monolayer of cells; and
collecting the confluent monolayer of cells.

15. The method of claim 14, wherein the specified shape is selected from the group consisting of a circle, an oval, an oval cross, a star, and a hand.

16. The method of claim 14, further comprising culturing the confluent monolayer for a sufficient time to allow the confluent monolayer to invaginate.

17. The method of claim 14, wherein the confluent monolayer is mechanically detached from the array spot.

18. The method of claim 14, wherein the confluent monolayer is cultured until the confluent monolayer spontaneously detaches from the array spot.

19. The method of claim 14, wherein the cell is selected from the group consisting of an induced pluripotent stem cell, a mesenchymal stem cell, an umbilical vein endothelial cell, a dermal fibroblast, a fibrosarcoma cell, an embryonic stem cell, an iPS IMR90-4 cell and combinations thereof.
